(19) United States
(12) Patent Application Publication
(10) Pub. No.: US 2006/0127413 A1
(43) Pub. Date: Jun. 15, 2006

(54) Recombinant mva strains as potential vaccines against p. falciparum malaria

(76) Inventors: Gerd Sutter, Munchen (DE); Hermann Bujard, Heidelberg (DE); Nicole Westerfeld, Heidelberg (DE); Miao Jun, Heidelberg (DE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/532,067

(22) PCT Filed: Sep. 26, 2003
(86) PCT No.: PCT/EP03/10723

§ 371 Date: Dec. 28, 2005

(30) Foreign Application Priority Data
Oct. 23, 2002 (DE) 102 49 390.1

(51) Int. Cl. A61K 39/12; C12N 7/00; C12N 15/86

(52) U.S. Cl. 424/199.1; 435/456; 435/235.1

(57) ABSTRACT

This invention relates to recombinant viruses based on MVA, which comprise at least one nucleic acid coding for a Plasmodium falciparum MSP-1 protein, a fragment or mutein of it. Furthermore, methods for the production of the recombinant viruses, virus-containing vaccines and the use of the recombinant viruses for the prophylaxis and/or therapy of malaria are provided.

### CROSS-REFERENCE

This application is a national stage filing under 35 U.S.C.§371 of International Patent Application Serial No. PCT/EP2003/010723, which was filed on Sep. 26, 2003 and which was published as WO 2004/038024 on May 6, 2004 which International Patent Application claims benefit of priority of German Patent Application no. 10249390.1, filed Oct. 23, 2002, which application is incorporated herein by reference in its entirety.

### FIELD OF THE INVENTION

This application is in the field of recombinant vaccinia virus, and in the field of vaccines to Plasmodium falciparum malaria.

### BACKGROUND OF THE INVENTION

The invention relates to the production of recombinant vaccinia viruses of the strain MVA (Modified Vaccinia Virus Ankara) for the recombinant production of the complete malaria antigen gp190/MSP-1 of the malaria pathogen Plasmodium falciparum as well as individual naturally occurring domains and parts thereof. Furthermore, the invention relates to the use of recombinant MVA which contain the synthetic DNA sequence of the malaria antigen and parts thereof integrated into the virus genome as vaccines for immunisation against malaria.

Malaria is one of the most dangerous infectious diseases in the world. According to estimates from the World Health Organisation (WHO) 400 to 900 million incidences of the disease are registered annually. According to information from the Multilateral Initiative against Malaria (MIM) between 700,000 and 2.7 million people die each year from the infection (MIM, 2001). In this respect 40% of the world's population in 99 countries are put at risk from malaria. The disease is caused by single-cell protozoa of the genus Plasmodium from the phylum Apicomplexa. There are four species which infect humans: Plasmodium malariae, responsible for Malaria quartana, Plasmodium vivax and Plasmodium ovale, both of which cause Malaria tertiana, and finally Plasmodium falciparum, the pathogen of Malaria tropica and responsible for almost all fatal infections.

It is again currently spreading to an increasing extent. This is primarily attributable to intensive resistance formation of the malaria pathogen which is promoted in that the medicaments used for the therapy are also recommended and used for prophylaxis. Apart from the search for new chemotherapeutics, research is concentrating on the development of vaccines, because in the course of malaria infections in humans, immunity mechanisms are applied, a fact which is expressed in an increased resistance to the plasmodia, as demonstrated in the development of various types of immunity in humans in regions where malaria epidemics prevail.

MSP-1 as Potential Vaccine

MSP-1, the main surface protein of merozoites, the invasive form of the blood phases of the malaria pathogen, is a 190-220 kDa protein. This protein is processed later during the development of the merozoites into smaller protein fragments, which can be present and isolated up to the invasion of erythrocytes due to the parasites anchored as a complex via a glycosylphosphatidylinositol anchor on the merozoite surface.

The sequences of the MSP-1 proteins of various P. falciparum strains fall into two groups, which have been named after two representative isolates Kᵢ and MAD20. Overall the protein consists of a number of highly preserved regions, of dimorphous regions, each of which can be assigned to one of the two relatively small oligomorphous blocks in the N-terminal part of the protein (FIG. 1; Tanabe et al., 1987).

The immunisation of mice with the protein purified from P. yoelii parasites protected the animals from the otherwise fatal infection (Holder and Freeman, 1981). Also the transfer of monoclonal antibodies against MSP-1 from P. yoelii provided protection in the mouse model (Majarian et al., 1984).

Apart from studies on mice, Saimiri and Aotus monkeys have also been immunised with native, immune-affinity purified MSP-1. In these tests the protein obtained from P. falciparum partially (Perrin et al., 1984) resp. completely (Siddiqui et al., 1987) protected against the ensuing infection with the parasite.

Purification of native material from Plasmodia is however expensive and cannot be used for production on a large scale. Therefore research into vaccines is concentrated on the development of recombinant vaccines.

For example, mice have been successfully immunised with MSP-1-19 purified from E. coli or Saccharomyces cerevisiae (Daly and Long, 1993; Ling et al., 1994; Tian et al., 1996; Hirunpetcharat et al., 1997), similarly as Mycobacterium bovis carrying MSP-1-19 (Matsumoto et al., 1999). Alternatively to immunisation with native or recombinant proteins, DNA coding for MSP-1-19 has also been used as a vaccine and protected immunised mice against infection with P. chabaudi (Wunderlich et al., 2000).

The immunisation of monkeys with recombinant MSP-1-19 and MSP-1-42 from P. falciparum provided partial protection (Kumar et al., 1995; Egan et al., 2000; Chang et al., 1996; Stowers et al., 2001). The interpretation of immunisation experiments on monkeys is however only conditionally possible, because a statistical evaluation of the results does not arise due to the low number of animals in the experiment.

In Phase I and II studies with MSP-1 fragments as vaccine their immunogeneity was also shown in humans. In this respect p19 from P. falciparum fused on a T-helper cell epitope of tetanus toxin (Keitel et al., 1999) and the MSP-1 blocks 3 and 4 (Saul et al., 1999; Genton et al., 2000) are involved.

Some epidemiological studies in endemic regions show with adults a correlation between antibody titers against MSP-1 and the immunity against malaria (Tolle et al., 1993; Riley et al., 1992; Riley et al., 1993).

These investigations together with the immunisation studies on animals prove that MSP-1 is a promising candidate for the development of a malaria vaccine.

Generally, these studies can be differentiated into two approaches; either purified material from parasites or material obtained in heterologic systems was used.

Both for functional investigations and also for use as a vaccine, proteins must be produced reproducibly in a good yield and high quality. MSP-1 can be purified from parasites, but this is only possible on a small scale and with great expense and therefore cannot be carried out for obtaining MSP-1 according to the stated criteria in this way.

Vaccinia viruses belong to the genus Orthopoxvirus in the branch Chordopoxyirinae. With the pox viruses complex viruses are involved which, with a double-strand DNA genome of about 200 kb and a size of 250×350 nm, are some of the largest known viruses. They consist of a cuboid shaped virion which is enclosed in a membrane envelope. In the host cell, replication and generation of the pox viruses takes place exclusively in the cytoplasm (for an overview: Moss et al., 1996). Here, Vaccinia viruses possess a very wide host cell spectrum and they infect almost all cells both from humans and also animals. In 1953 Anton Mayr isolated and purified the dermovaccinia strain Chorioallantois Vaccinia Ankara (CVA). This virus was propagated further with continuous passage on chicken embryo fibroblasts and an attenuated virus was obtained, which did not show any further virulence in animals and humans (Stickl et al., 1974). Irrespective of this, the virus could be further used for immunoprophylaxis against diseases caused by orthopox viruses in humans and animals (Stickl et al., 1974). This virus was named Modified Vaccinia Virus Ankara (MVA) after its location of origin.

Considered on a molecular genetic level, during over 570 passages on chicken embryo fibroblasts the virus lost 31 kb of DNA sequence of its genome, principally in the form of six larger deletions, including at least two genes which determine the host spectrum and therefore the capability of the virus to replicate (Meyer et al., 1991). During MVA infection in most of the cells originating in mammals, including human cells, the formation of infectious virus particles is blocked only very late in the infection cycle at the phase of virion formation, i.e. viral genes under the control of promoters, both for the early and also intermediate and late transcription, can be expressed even in non-permissive cells. This differentiates MVA from other attenuated and replication-deficient pox viruses, such as for example, Vaccinia virus NYVAC or canary pox virus ALVAC, the infection of which is interrupted in most cells originating from mammals already before the viral DNA replication (Tartaglia et al., 1992; Sutter and Moss, 1992).

In the development of malaria vaccines various recombinant Vaccinia viruses have been used and in this respect replication-competent viruses of the type Western Reserve and Copenhagen and attenuated viruses of the types NYVAC, ALVAC or COPAC have been used (Kaslow, et al., 1991; Etlinger and Altenburger, 1991; Aidoo et al., 1997; Allsopp et al., 1996).

In connection with the attenuated Vaccinia virus MVA, only recombinant viruses have been described which carry CSP from the rodent parasite Plasmodium berghei as malaria antigen (Schneider et al., 1998; Plebanski et al., 1998; Degano et al., 1999; Gilbert et al., 1999).

Furthermore, recombinant Vaccinia viruses are described, which contain an MSP-1 coding sequence. The authors of one publication state that they have integrated the native MSP-1 coding sequence into the genome of the virus type Western Reserve, but do not support this statement experimentally (no restriction analyses, PCR, Northern Blot and Western Blot analyses, etc.). An immunisation of Saimiri monkeys with these recombinant viruses did not lead to the formation of MSP-1 specific antibodies and moreover also remained without measurable influence on the humoral immune response against MSP-1 after a P. falciparum infection (Pye et al., 1991).

In a further publication the vector NYVAC-Pf7 is described, which, among other things, expresses msp-1 of P. falciparum. The sera of two of the six immunised Rhesus monkeys show in the Western Blot analysis none of the signals against native MSP-1 detectable in the publication. The signals from three further animals detect solely parts of the protein complex and only the serum of one of the immunised animals detects a broader band spectrum. Overall however, these signals also appear to be weak. There is no reference to quantitative analyses on MSP-1 specific antibodies by ELISA (Tine et al., 1996).

In human experiments of the Phase I/IIa with NYVAC-Pf7 neither a cellular immune response against MSP-1 nor a humoral immune response verifiable by ELISA was proven (Ockenhouse et al., 1998).

In contrast to this, Tine et al. (1996) verify intact MSP-1 in the Western Blot analysis, whereby it appears to be a contradiction that MSP-1 is transported out of the cell by P. falciparum signal sequences (cf. publications from Moran and Caras, 1994, Yang et al., 1997).

### SUMMARY OF THE INVENTION

This invention relates to recombinant viruses based on MVA, which comprise at least one nucleic acid coding for a Plasmodium falciparum MSP-1 protein, a fragment or mutein of it. Furthermore, methods for the production of the recombinant viruses, virus-containing vaccines and the use of the recombinant viruses for the prophylaxis and/or therapy of malaria are provided.

### BRIEF DESCRIPTION OF THE DRAWINGS

FIG. 1 depicts the primary structure of the MSP-1 derived from the FCB-1 and MDA20 strains of P. falciparum.

FIG. 2 depicts verification of MSP-1D-42 and MSP-1D-38/42 in HeLa cells infected with recombinant MVA using immunoblot.

FIGS. 3A and 3B depict development of a humoral response after three immunizations with rMVA-msp1d/42S or rMVA-msp1d/42A and one immunization with MSP-1 D-HX42 from E. coli.

FIG. 4 depicts development of a humoral immune response after immunizations with rMVA-msp1d/S or rMVA-msp1d/83+30/38+42A in combination with immunization with MSP-1D from E. coli.

### DETAILED DESCRIPTION OF THE INVENTION

The object of this invention is therefore to provide a recombinant Vaccinia virus that is capable of

containing stably integrated DNA sequences, which code for MSP-1 of P. falciparum or partial sections of it,

expressing these sequences efficiently and reproducibly and therefore

produce MSP-1 protein in secreted or surface-anchored form to

immunise a host and thereby

cause a cellular and humoral immune response.

The object of the invention is solved by the provision of a recombinant MVA virus, which is capable of infection, replication and expression of MSP-1 in a host. Furthermore, methods for the production and use of the recombinant virus are given.

According to the invention, the expression “virus based on MVA” signifies a virus derived from MVA, which exhibits one or more mutations in non-essential regions of the virus genome. The essential regions of the MVA virus are in this regard all genome sections of the MVA virus, which are necessary for receiving the viral gene expression and the capability of the MVA virus for propagation. This includes, for example, the gene sequences coding for viral RNA polymerase subunits or the viral DNA polymerase. Preferably the virus based on MVA is the MVA virus.

The Vaccinia system NYVAC-Pf7, known from the state of the art, is based on the basic virus NYVAC, which originally comes from the Copenhagen Vaccinia virus strain and was attenuated by the targeted deletion of 18 open reading frames. However, with NYVAC in mammal cells the DNA replication is blocked (Tartaglia et al., 1992), whereas with MVA the virus assembly is suppressed. This has the advantage that in contrast to NYVAC in MVA, late gene suppression occurs, which can be used for the expression of recombinant genes. MVA can therefore both preferentially induce a cytotoxic T-cell response during the early transcription phase as well as stimulate the humoral branch of the immune response due to the high protein expression during the late phase.

MVA, which has already been extensively employed during the pox protection vaccination campaign, is regarded as a very safe virus for vaccination on humans (Stickl et al., 1974).

According to the invention, a recombinant virus based on MVA is provided, comprising at least one nucleic acid encoding a P. falciparum MSP-1 protein, a fragment or mutein thereof.

The MSP-1 amino acid sequence can be obtained from publicly accessible data bases. 3D7 (MAD20 isolate): CAA84556; FCB-1 (K1 isolate): CAB36903, both from the NIH data base on the internet at ncbi.nlm.nih.gov.

Particularly preferred is the nucleic acid encoding MSP-1 protein, a nucleic acid reduced in its AT content, as described in DE 19640817 A1, the disclosure content of which is included hereby. In particular a nucleic acid is preferred which is derived from the P. falciparum MSP-1 sequence and in which a large part of the plasmodium codons has been replaced such that the codon frequency of the synthetic gene corresponds to the human one without the amino acid sequence being changed.

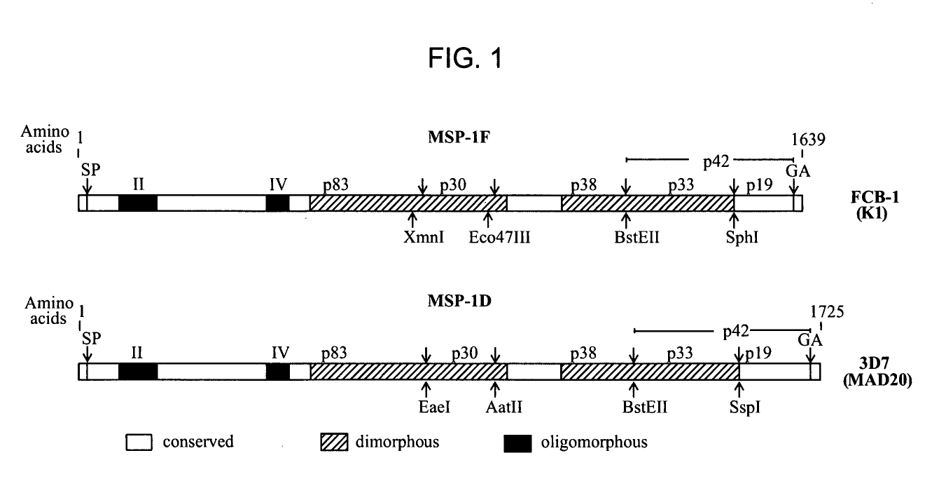

According to a preferred embodiment, the MSP-1 protein is the MSP-1 of the isolate 3D7, the MSP-1 protein which is designated in the following as “MSP-1D”. Alternatively, this can be the MSP-1 protein of the FCB1 strain, which is designated as “MSP-1F” in the following. The MSP-1 protein preferably comprises also fragments of these two forms of MSP-1. Especially preferred in this respect are, alone or in combination, the fragments of MSP-LF p83, p30, p38, p33 and p19. Especially preferred, alone or in combination, whereby here similarly combinations with MSP-1F fragments are included, are fragments of MSP-1D, in particular p83, p30, p38, p33 and p19. In particular the combinations of p83 and p30 as well as p38 and p42 are preferred. The position of the fragments is in this respect shown in FIG. 1. Furthermore, the fragment p42 of both MSP-1 forms is also included.

The MSP-1 protein can also be a mutein of the P. falciparum MSP-1 sequence, which is differentiated from the MSP-1 sequence of the wild type by addition, deletion, insertion, inversion or substitution of one or more amino acids.

In a preferred embodiment the virus according to the invention comprises a promoter suitable for expression, whereby the sequence encoding msp-1 is under the control of the promoter. The promoter can in this respect be an MVA promoter, whereby the promoter can be an early, intermediate or late gene promoter or a combination of them. However, non-MVA promoters are also included which are capable of expression in the expression system used. In this respect, both constitutive as well as inducible promoters can be used. For large-scale protein production in this respect, a strong Vaccinia virus promoter, such as the synthetic late or early/late promoter or the HybridVaccinia/T7 polymerase system can be used; for the induction of MHC Class I restricted cytotoxic T-cell response in vivo a naturally occurring early or tandem early/late promoter can be used; furthermore the E. coli lac repressor/operator system or the HybridVaccinia/T7 system can be used for the initiation of the gene expression; (Methods in Molecular Biology, Volume 62, published by Rocky S. Tuan, Humana Press, Broder and Earl, page 176, with further verification).

According to a further preferred embodiment, the virus also comprises a selection marker. The selection marker is in this respect suitable for the selection and/or for screening in a known manner. Suitable selection markers comprise in this respect for example the E. coli lacZ system, the selection system using the E. coli xanthine-guanine phosphoribosyl transferase (XGPRT) gene. In addition selection methods can be used which modify the host cell specificity of the viruses (Staib et al., 2000). Other selection markers known in the state of the art can be used.

According to a further preferred embodiment, the nucleic acid is fused at the 5′ end with a nucleotide sequence encoding a signal peptide sequence. As known from the state of the art, the signal and anchor sequences from P. falciparum are not detected with expression in mammalian cells or are not correctly processed (Moran and Caras, 1994; Burghaus et al., 1999; Yang et al., 1997).

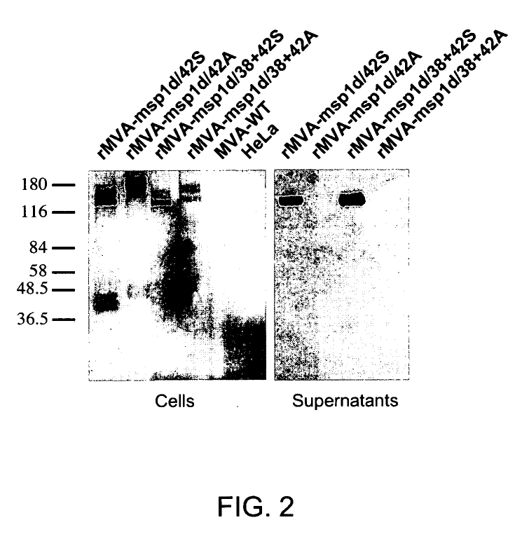

The problem of the selective control of the intracellular gating is solved by the use of the signal sequences of the human “Decay Accelerating Factor” (DAF) (FIG. 2). Suitable signal peptide sequences are specific for higher eukaryotes. Examples of such signal sequences apart from those of the decay accelerating factor are immunoglobulins or signal peptides of various growth factors and cytokines (von Heijne, 1985). According to a preferred embodiment the signal peptide sequence controls the secretion of the gene product, for example cytokines, antibodies, etc.

Furthermore, signal sequences are preferred which lead to GPI anchoring of the C terminus of the gene product on the cell surface, as with the human DAF. Alternatively, peptide sequences are preferred which control the membrane-compatible localisation of the gene product, as in the case of immunoglobulins of the M isotype or of the Vesicular Stomatitis virus G protein.

According to a further preferred embodiment the virus can also contain suitable splice donor and splice acceptor sites, so that an appropriately spliced mRNA arises, which is suitable for translation within the individual to be treated. The nucleic acid can in addition contain a sequence which is suitable as a ribosome binding site.

According to a further embodiment of the invention, a method for the production of a recombinant virus is provided, whereby the method comprises the steps:

a) Transfecting of a eukaryotic host cell with a transfer vector, whereby the transfer vector

i) comprises a Plasmodium falciparum MSP-1, a nucleic acid coding for a fragment or a mutein thereof, whereby the mutein is differentiated by addition, deletion, insertion, inversion or substitution of one or more amino acids of the MSP-1 sequence, and optionally comprises the coding sequence for a selection marker; and comprises DNA sequences, which act as promoters for the transcription control of the coding sequences;

ii) the nucleic acid according to i) is flanked by MVA sequences 5′ and/or 3′, whereby the sequences are suitable for the homologous recombination with genomic MVA-DNA in the host cell;

b) infection with a virus based on MVA, preferably MVA;

c) cultivation of the host cell under conditions suitable for homological recombination; and

d) isolation of the recombinant virus based on MVA.

Preferably, the host cell is selected from RK13 (rabbit kidney cells), BHK21 (baby hamster kidney cells) or primary CEF (chicken embryo fibroblast cells).

The transfer vector can be a typical Vaccinia virus transfer vector, which for example is selected from pGS20, pSC59, pMJ601, pSC65, pSC11, pCF11 and pTKgptF1s or vectors which are derived from them; refer to Methods in Molecular Biology, Volume 62, see above, Broder and Earl, page 176 and other references mentioned in it, in particular Earl, Cooper and Moss (1991) in Current Protocols in Molecular Biology (Ausubel et al.), Wiley Interscience, New York, pages 16.15.1-16.18.10. The transfection occurs according to conditions known in the state of the art.

The nucleic acid can in this respect have the modifications stated for the nucleic acid of the virus.

The nucleic acid according to i) is flanked by MVA sequences or complementaries of it 5′ and/or 3′; preferred flanking MVA sequences are DNA sequences in each case 5′ and 3′ of naturally occurring deletion sites in the MVA genome, e.g. deletion sites II, III or VI as described in Meyer H., Sutter G., Mayr A. (1991), J Gen Virol 72, 1031-1038 or as can be seen from the complete genome sequence of the MVA virus (Antoine et al. 1998, Virology 244, 365-396). Preferably the transfer vector comprises furthermore a selection marker such as for example an antibiotic resistance or a metabolism marker. Principally however, all selection markers known in the state of the art are comprised.

For the efficient homologous recombination the MVA-DNA sequences flanking the nucleic acid to be inserted should exhibit a length in each case of at least 0.5 kb.

The host cell is transfected with the transfection vector according to known methods. The infection with MVA occurs according to standard conditions (Staib et al., 2000).

The isolation of the recombinant MVA virus occurs based on the selection marker within the sequence according to alternative (i). The recombinant MVA virus can be obtained either directly from the lysate of the cultivated host cells or from the culture supernatant.

According to a further embodiment, a vaccine is made available which comprises:

a) the recombinant virus according to the invention; and

b) a pharmacologically compatible carrier.

Pharmacologically compatible carriers are in this respect all carriers and dilution agents known in the state of the art. If a certain type of application is intended, the pharmacologically compatible carrier can be selected or modified in a known manner.

The vaccine can be administered subcutaneously, intramuscularly, intravenously, transdermally, intraperitoneally or orally. The vaccine is specified for prophylaxis and/or therapy of malaria in humans and animals.

According to a preferred embodiment, the vaccine can furthermore contain MSP-1, a fragment or a mutein thereof, which is differentiated by addition, deletion, insertion, inversion or substitution of one or more amino acids from the Plasmodium falciparum MSP-1 sequence, and/or a nucleic acid coding it. More preferably, the MSP-1 protein is in this respect produced recombinantly, in particular recombinantly in E. coli. The nucleic acid coding for MSP-1 or a mutein of it is preferably one which is reduced with regard to its AT content. Especially preferred is a nucleic acid such as described in DE-19640817 A1, with which in particular Plasmodium falciparum codons are replaced by human codons without changing the amino acid sequence.

Where the vaccine comprises both the recombinant virus as well as MSP-1, a fragment or a mutein of it or the coding nucleic acid, then the vaccine can be provided in kit form. It is therefore suitable for simultaneous, sequential or separate administration of the two components of the vaccine.

### EXAMPLES

The following examples explain the invention, but do not restrict its object.

FIG. 1: Primary Structure of the MSP-1 Derived from the FCB-1 and MAD20. Strains of P. falciparum.

The arrows above the sequence identify the processing sites of the native proteins (Holder et al., 1987), which divide MSP-1 into the fragments p83, p30, p38 and p42, which are anchored as complexes on the parasite surface. In the second process stage p42 is split to form p33 and p19. The arrows below the illustrations designate the uniquely occurring endonuclease cleavage sites of the synthetic DNA sequences.

Abbreviations: SP=Signal Peptide, GA=GPI Anchor

HeLa cells were infected with rMVA-msp1d/38+42S or rMVA-msp1d/38+42A and then fixed. Some cells were permeabilized with Triton X-100 after fixing. The cells thus treated were incubated with mAk 5.2 as the first antibody, which recognises a conformational epitope specific for MSP-1 in the C-terminal part of the MSP-1 fragment p19 and a polyclonal serum, which recognises the ER protein Sec61beta (anti-ER marker). These were colour labeled using Cy3 conjugated anti-mouse IgG (detects mAk 5.2) or FITC conjugated anti-rabbit IgG (detects anti-Sec61beta) and then analysed in the confocal microscope. If the cells were infected with rMVA-msp1d/38+42S or rMVA-msp1d/38+42A and permeabilised, then the signal can be colocalised for MSP-1 D-38/42 (mAk 5.2) with the ER marker. If in contrast the cells remain intact, MSP-1D-38/42 is only detected in the case of infection with rMVA-msp1d/38+42A on the surface of the infected cells. The ER marker here acts as a control for the intact condition of the cell membrane.

Abbreviations: ER=Endoplasmic Recticulum; mAK=monoclonal Antibody.

FIG. 2: Verification of MSP-1D-42 and MSP-1D-38/42 in HeLa Cells Infected with Recombinant MVA Using Immunoblot

HeLa cells were infected with rMVA-msp1d/42S, rMVA-msp1d/42A, rMVA-msp1d/38+42S or rMVA-msp1d/38+42A and incubated overnight. Samples of the supernatant and the cellular fraction were separated using SDS-gel electrophoresis under non-reducing conditions, transferred to a PVDF membrane and verified using mAb 5.2 primary antibodies. Only chimera from the DAF signal sequence and the corresponding MSP-1D fragments can be verified in the supernatants of the infected cells, whereas the intracellular expression in all cells infected with recombinant MVA supplies a signal.

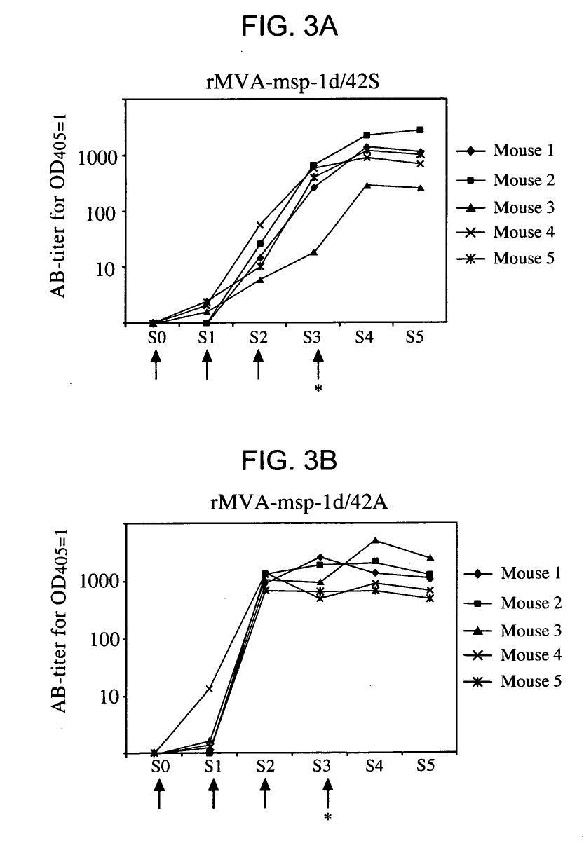

FIGS. 3A and 3B: Development of the Humoral Immune Response After Three Immunisations with rMVA-msp1d/42S or rMVA-msp1d/42A and One Immunisation with MSP-1D-HX42 from E. coli.

In FIG. 3A the analysis of the humoral immune response is shown using ELISA with recombinantly produced MSP-1 D-HX42, purified from E. coli as antigen. The curves illustrate the p42 specific antibody development, measured on the OD₄₀₅=1. The mice were in each case immunised at intervals of three weeks with 10⁶ IU (first immunisation, simultaneous with blood withdrawal S0) or 10⁸ IU (1st and 2nd boost, simultaneous with S1 and S2) of rMVA-msp1d/42S. In addition the mice were injected subcutaneously after a further four weeks each with 5 μg of MSP-1D-HX42 from E. coli in the incomplete Freund's adjuvant (one week after the blood withdrawal S3). S0 to S5 represent the times of the blood withdrawal and here the blood S0 to S3 was in each case taken at intervals of three weeks and the withdrawals of S4 and S5 occurred in each case at intervals of four weeks. The arrows mark the times of the immunisations. The asterisk marks the fourth immunisation with MSP-1D-HX42 from E. coli.

FIG. 3B shows the same analysis for the immunisation with rMVA-msp1d/42A.

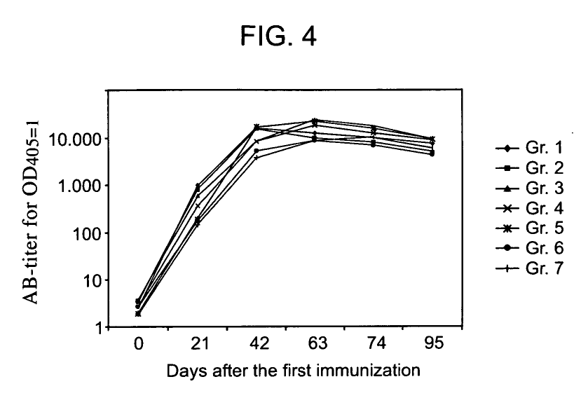

FIG. 4: Development of the Humoral Immune Response After Immunisations with rMVA-msp1d/S or rMVA-msp1d/83+30/38+42A in Combination with Immunisation with MSP-1D from E. coli.

The humoral immune response was determined using ELISA under application of recombinantly produced MSP-1D purified from E. coli as antigen. As in FIGS. 3A and 3B, the curves show the MSP-1D specific antibody development, measured on the OD₄₀₆=1.

The mice were in each case immunised at intervals of three weeks (labelled in the illustration by arrows). The immunisation strategies allocated to the groups were composed as follows:

Gr. 1: 20 μg of MSP-1D (Day 0), 10⁸ IU of rMVA-msp1d/S (Day 21), 5 mice

Gr. 2: 20 μg of MSP-1D (Day 0), 10⁸ IU of rMVA-msp1d/A (Day 21), 5 mice

Gr. 3: 20 μg of MSP-1D (Day 0), 10⁸ IU of rMVA-msp1d/S (Day 21), 20 μg of MSP-1D (Day 42), 10 mice

Gr. 4: 20 μg of MSP-1D (Day 0), 10⁸ IU of rMVA-msp1d/S (Day 21), 10⁸ IU of rMVA-msp1d/S (Day 42), 10 mice

Gr. 5: 20 μg of MSP-1D (Day 0), 10⁸ IU of rMVA-msp1d/83+30/38+42A (Day 21), 20 μg of MSP-1D (Day 42), 9 mice

Gr. 6: 20 μg of MSP-1D (Day 0), 10⁸ IU of rMVA-msp1d/83+30/38+42A (Day 21), 10⁸ IU of rMVA-msp1d/83+30/38+42A (Day 42), 10 mice

Gr. 7: 20 μg of MSP-1D (Day 0), 10⁸ IU of rMVA-msp1d/A (Day 21), 20 μg of MSP-1D (Day 42), 5 mice

In the following, recombinant viruses, which lead to the production of MSP-1 in its surface-anchored form, are labelled with “A” and those which lead to the secretion of MSP-1 with “S”.

TABLE 1
Complete list of the virus-constructs produced
in the scope of the invention:

| | rMVA-msp1d | rMVA-msp1f |
|---|---|---|
| Secreted MSP-1 | rMVA-msp1d/S | |
| | rMVA-msp1d/83S | |
| | rMVA-msp1d/83 + 30S | |
| | rMVA-msp1d/42S | |
| | rMVA-msp1d/38 + 42S | |
| Surface- | rMVA-msp1d/A | rMVA-msp1f/A |
| anchored MSP-1 | | |
| | rMVA-msp1d/83A | rMVA-msp1f/83 + |
| | | 30/38 + 42A |
| | rMVA-msp1d/83 + 30A | rMVA-msp1f/38 + |
| | | 42A |
| | rMVA-msp1d/42A | |
| | rMVA-msp1d/38 + 42A | |
| | rMVA-msp1d/83 + 30/38 + 42A | |

The production of MSP-1 or the fragments and the localisation of the proteins in the infected cell was proven using confocal microscopy and is illustrated as an example of the infection of HeLa cells by rMVA-msp1d/38+42S and rMVA-msp1d/38+42A.

The secretion of all MSP-1 variants from cells infected with recombinant MVA was verified using immunoblot analyses of cellular supernatants and is illustrated here as an example for rMVA-msp1d/42S and rMVA-msp1d/38+42S (FIG. 2).

Then the recombinant MVA were examined in immunisation experiments on mice for their immunogenic effect with regard to the humoral immune response. Here, for msp-1 recombinant MVA induced high antibody titers against the parasite protein which was determined using ELISA. The p42 specific antibody titers and the observed, different immunisation potential of the surface-anchored or secreted proteins produced by the recombinant MVA is illustrated as an example of immunisations with rMVA-msp1d/42S and rMVA-msp1d/42A (FIGS. 3A and 3B).

### REFERENCES

Aidoo, M., Ulvani, A., Whittle, H. C, Hill, A. V., and Robson, K. J. (1997). Recombinant vaccinia viruses for the characterization of Plasmodium falciparum-specific cytotoxic T lymphocytes: recognition of processed antigen despite limited re-stimulation efficacy. Int Immunol 9, 731-7.
Allsopp, C. E., Plebanski, M., Gilbert, S., Sinden, R. E., Harris, S., Frankel, G., Dougan, G., Hioe, C., Nixon, D., Paoletti, E., Layton, G., and Hill, A. V. (1996). Comparison of numerous delivery systems for the induction of cytotoxic T lymphocytes by immunization. Eur J Immunol 26, 1951-9.
Antoine, G., Scheiflinger, F., Dorner, F, and Falkner, F. G. (1998). The complete genomic sequence of the modified vaccinia Ankara strain: Comparison with other Orthopoxviruses. Virology 244, 365-396.
Chang, S. P., Case, S. E., Gosnell, W. L., Hashimoto, A., Kramer, K. J., Tarn, L. Q., Hashiro, C. Q., Nikaido, C. M., Gibson, H. L., Lee-Ng, C. T., Barr, P. J., Yokota, B. T., and Hut, G. S. (1996). A recombinant baculovirus. 42-kilodalton C-terminal fragment of Plasmodium falciparum merozoite surface protein 1 protects Aotus monkeys against malaria. Infect Immun 64, 253-61.
Daly, T. M. and Long, C. A. (1993). A recombinant 15-kilodalton carboxyl-terminal fragment of Plasmodium yoelii yoelii 17XL merozoite surface protein 1 induces a protective immune response in mice. Infect Immun 61, 2462-7.
Degano, P., Schneider, J., Hannan, C. M., Gilbert, S. C., and Hill, A. V. (1999). Gene gun intradermal DNA immunization followed by boosting with modified Vaccinia virus Ankara: enhanced CD8+ T cell immunogenicity and protective efficacy in the influenza and malaria models. Vaccine 18, 623-32.
Egan, A. F., Blackman, M. J., and Kaslow, D. C. (2000). Vaccine efficacy of recombinant Plasmodium falciparum merozoite surface protein 1 in malaria-naive, -exposed, and/or -rechallenged Aotus vociferans monkeys. Infect Immun 68, 1418-27.
Etlinger, H. M., and Altenburger, W. (1991). Overcoming inhibition of antibody responses to a malaria recombinant vaccinia virus caused by prior exposure to wild type virus. Vaccine 9, 470-2.
Genton, B., Al-Yaman, F., Anders, R., Saul, A., Brown, G., Pye, D., Irving, D. O., Briggs, W. R., Mai, A., Ginny, M., Adiguma, T., Rare, L., Giddy, A., Reber-Liske, R., Stuerchler, D., and Alpers, M. P. (2000). Safety and immunogenicity of a three-component blood-stage malaria vaccine in adults living in an endemic area of Papua New Guinea. Vaccine 18, 2504-11.
Gilbert, S. C., Schneider, J., Plebanski, M., Hannan, C. M., Blanchard, T. J., Smith, G. L., and Hill, A. V. (1999). Ty virus-like particles, DNA vaccines and Modified Vaccinia Virus Ankara; comparisons and combinations. Biol Chem 380, 299-303.
Hirunpetcharat, C., Tian, J. H., Kaslow, D. C., van Rooijen, N., Kumar, S., Berzofsky, J. A., Miller, L. H., and Good, M. F. (1997). Complete protective immunity induced in mice by immunization with the 19-kilodalton carboxyl-terminal fragment of the merozoite surface protein-1 (MSP1[19]) of Plasmodium yoelii expressed in Saccharomyces cerevisiae: correlation of protection with antigen-specific antibody titer, but not with effector CD4+ T cells. J Immunol 159, 3400-11.
Holder, A. A., and Freeman, R. R. (1981). Immunization against blood-stage rodent malaria using purified parasite antigens. Nature, 294, 361-364.
Holder, A. A., Sandhu, J. S., Hillman, Y., Davey, L. S., Nicholls, S. C., Cooper, H., and Lockyer, M, J. (1987). Processing of the precursor to the major merozoite surface antigens of Plasmodium falciparum. Parasitology 94, 199-208.
Kaslow, D. C., Isaacs, S. N., Quakyi, I. A., Gwadz, R. W., Moss, B., and Keister, D. B. (1991). Induction of Plasmodium falciparum transmission-blocking antibodies by recombinant vaccinia virus. Science 252, 1310-1313.
Keitel, W. A., Kester, K. E., Atmar, R. L., White, A. C., Bond, N. H., Holland, C. A., Krzych, U., Palmer, D. R., Egan, A., Diggs, C., Ballou, W. R., Hall, B. F., and Kaslow, D. (1999). Phase I trial of two recombinant vaccines containing the 19 kd carboxy terminal fragment of Plasmodium falciparum merozoite surface protein 1 (msp-1 (19)) and T helper epitopes of tetanus toxoid. Vaccine 18, 531-9.
Kumar, S., Yadava, A., Keister, D. B., Tian, J. H., Ohl, M., Perdue-Greenfield, K. A., Miller, L. H., and Kaslow, D. C. (1995). Immunogenicity and in vivo efficacy of recombinant Plasmodium falciparum merozoite surface protein-1 in Aotus monkeys. Mol Med 1, 325-32. Ling, I. T., Ogun, S. A., and Holder, A. A. (1994). Immunization against malaria with a recombinant protein. Parasite Immunol 16, 63-7.
Majarian, W. R., Daly, T, M., Weidanz, W. P., and Long, C. A. (1984). Passive immunization against murine malaria with an IgG3 monoclonal antibody. J. Immunol. 132, 3131-3137.
Matsumoto, S., Yukitake, H., Kanbara, H., and Yamada, T. (1999). Long-lasting protective immunity against rodent malaria parasite infection at the blood stage by recombinant BCG secreting merozoite surface protein-1. Vaccine 18, 832-4.
Meyer, H., Sutter, G., and Mayr, A. (1991). Mapping of deletions in the genome of the highly attenuated vaccinia virus MVA and their influence on virulence. J Gen Virol 72, 1031-8.
Moran, P., and Caras, I. W. (1994). Requirements for glycosylphosphatidylinositol attachment are similar but not identical in mammalian cells and parasitic protozoa. J Cell Biol 125, 333-43.
Moss, B., Carroll, M. W., Wyatt, L. S., Bennink, J. R., Hirsch, V. M., Goldstein, S., Elkins, W. R., Fuerst, T. R., Lifson, J. D., Piatak, M., Restifo, N. P., Overwijk, W., Chamberlain, R., Rosenberg, S. A., and Sutter, G. (1996). Host range restricted, non-replicating vaccinia virus vectors as vaccine candidates. Adv Exp Med Biol 397, 7-13.
Ockenhouse, C. F., Sun, P. F., Lanar, D. E., Wellde, B. T., Hall, B. T, Kester, K., Stoute, J. A., Magill, A., Krzych, U., Farley, L., Wirtz, R. A., Sadoff, J. C., Kaslow, D. C., Kumar, S., Church, L. W., Crutcher, J. M., Wizel, B., Hoffman, S., Lalvani, A., Hill, A. V., Tine, J. A., Guito, K. P., de Taisne, C., Anders, R., Ballou, W. R., and et al. (1998). Phase I/IIa safety, immunogenicity, and efficacy trial of NYVAC-Pf7, a pox-vectored, multiantigen, multistage vaccine candidate for Plasmodium falciparum malaria. J Infect Dis 177, 1664-73.
Perrin, L. H., Merkli, B., Loche, M., Chizzolini, C., Smart, J., and Richle, R. (1984). Antimalarial immunity in Saimiri monkeys. Immunization with surface components of asexual blood stages. J Exp Med 160, 441-451.
Plebanski, M., Gilbert, S. C., Schneider, J., Hannan, C. M., Layton, G., Blanchard, T., Becker, M., Smith, G., Butcher, G., Sinden, R. E., and Hill, A. V. (1998). Protection from Plasmodium berghei infection by priming and boosting T cells to a single class I-restricted epitope with recombinant carriers suitable for human use. Eur J Immunol 28, 4345-55.
Pye, D., Edwards, S. J., Anders, R. F., O'Brien, C. M., Franchina, P., Corcoran, L. N., Monger, C., Peterson, M. G., Vandenberg, K. L., Smythe, J. A., Westley, S. R., Coppel, R. L., Webster, T. L., Kemp, D. J., Hampson, A. W., and Langford. C. J. (1991). Failure of recombinant vaccinia viruses expressing Plasmodium falciparum antigens to protect Saimiri monkeys against malaria. Infect Immun 59, 2403-2411.
Riley, E. M., Allen, S. J., Wheeler, J. G., Blackman, M. J., Bennett, S., Takacs, B., Schonfeld, H. J., Holder, A. A., and Greenwood, B. M. (1992). Naturally acquired cellular and humoral immune responses to the major merozoite surface antigen (PfMSP1) of Plasmodium falciparum are associated with reduced malaria morbidity. Parasite Immunol 14, 321-337.
Riley, E. M., Morris-Jones, S., Blackman, M. J., Greenwood, B. M., and Holder, A. A. (1993). A longitudinal study of naturally acquired cellular and humoral immune responses to a merozoite surface protein (MSP1) of Plasmodium falciparum in an area of seasonal malaria transmission. Parasite Immunol 15, 513-524.
Saul, A., Lawrence, G., Smillie, A., Rzepczyk, C. M., Read, C., Taylor, D., Anderson, K., Stowers, A., Kemp, R., Allworth, A., Anders, R. F., Brown, G. V., Pye, D., Schoofs, P., Irving, D. O., Dyer, S. L., Woodrow, G. C., Briggs, W. R., Reber, R., and Sturchler, D. (1999). Human phase I vaccine trials of 3 recombinant asexual stage malaria antigens with Montanide ISA720 adjuvant. Vaccine 17, 3145-59.
Schneider, J., Gilbert, S. C., Blanchard, T. J., Hanke, T., Robson, K. J., Hannan, C. M., Becker, M., Sinden, R., Smith, G. L., and Hill, A. V. (1998). Enhanced immunogenicity for CD8+ T cell induction and complete protective efficacy of malaria DNA vaccination by boosting with modified vaccinia virus Ankara. Nat Med 4, 397-402.
Siddiqui, W. A., Tarn, L. Q., Kramer, K. J., Hui, G. S.,. Case, S. E., Yamaga, K. M., Chang. S. P., Chan, E. B., and Kan, S. C. (1987). Merozoite surface coat precursor protein completely protects Aotus monkeys against Plasmodium falciparum malaria. Proc Natl Acad Sci USA 84, 3014-3018.
Staib, C., Drexler, I., Ohlmann, M., Wintersperger, S., Erfle, V., and Sutter, G. (2000). Transient host range selection for genetic engineering of modified vaccinia virus Ankara. Biotechniques 28, 1137-42, 1144-6, 1148.
Stickl, H., Hochstein-Mintzel, V., Mayr, A., Huber, H. C., Schafer, H., and Holzner. A. (1974). [MVA vaccination against smallpox: clinical tests with an attenuated live vaccinia virus strain (MVA) (author's transl.)]. Dtsch Med Wochenschr 99, 2386-92.
Stowers, A. W., Cioce, V., Shimp, R. L, Lawson, M., Hui, G., Muratova, O., Kaslow, D. C., Robinson, R., Long, C. A., and Miller, L. H. (2001). Efficacy of two alternate vaccines based on Plasmodium falciparum merozoite surface protein 1 in an Aotus challenge trial. Infect Immun 69, 1536-46.
Sutter, G., and Moss, B. (1992). Nonreplicating vaccinia vector efficiently expresses recombinant genes. Proc Natl Acad Sci USA 89, 10847-51.
Tanabe, K., Mackay, M., Goman, M., and Scaife, J. G. (1987). Allelic dimorphism in a surface antigen gene of the malaria parasite Plasmodium falciparum. J Mol Biol 195, 273-287.
Tartaglia, J., Perkus, M. E., Taylor, J., Norton, E. K., Audonnet, J. C., Cox, W. I., Davis, S. W., van der Hoeven, J., Meignier, B., Riviere, M., and et al. (1992). NYVAC: a highly attenuated strain of vaccinia virus. Virology 188, 217-32.
Tian, J. H., Miller, L. H., Kaslow, D. C., Ahlers, J., Good, M. F., Alling, D. W., Berzofsky, J. A., and Kumar, S. (1996). Genetic regulation of protective immune response in congenic strains of mice vaccinated with a subunit malaria vaccine. J Immunol 157, 1176-83.
Tine, J. A., Lanar, D. E., Smith, D. M., Wellde, B. T., Schultheiss, P., Ware, L. A., Kauffman, E. B, Wirtz, R. A., De Taisne, C., Hui, G. S., Chang, S. P., Church, P., Hollingdale, M. R., Kaslow, D. C., Hoffman, S., Guito, K. P., Ballou, W. R., Sadoff J. C., and Paoletti, E. (1996). NYVAC-Pf7: a poxvirus-vectored, multiantigen, multistage vaccine candidate for Plasmodium falciparum malaria, Infect Immun 64, 3833-44.
Tolle, R., Fruh, K., Doumbo, O., Koita, O., N'Diaye, M., Fischer, A., Dietz, K, and Bujard, H. (1993). A prospective study of the association between the human humoral immune response to Plasmodium falciparum blood stage antigen gp190 and control of malarial infections. Infect Immun 61, 40-7.
von Heijne, G. (1985). Signal sequences. The limits of variation. J Mol Biol 184, 99-105.
Wunderlich, G., Moura, I. C., and del Portillo, H. A. (2000). Genetic immunization of BALB/c mice with a plasmid bearing the gene coding for a hybrid merozoite surface protein 1-hepatitis B virus surface protein fusion protects mice against lethal Plasmodium chabaudi chabaudi PC 1 infection. Infect Immun 68, 5839-45.
Yang, S., Carroll, M. W., Torres-Duarte, A. P., Moss, B., and Davidson, E. A. (1997). Addition of the MSA1 signal and anchor sequences to the malaria merozoite surface antigen 1 C-terminal region enhances immunogenicity when expressed by recombinant vaccinia virus. Vaccine 15, 1303-13.

1. A recombinant Modified Vaccinia Vaccine Ankara (MVA) virus comprising at least one nucleic acid coding for a Plasmodium falciparum merozoite surface protein-1 (MSP-1) protein or a fragment or mutein thereof.

2. The recombinant MVA virus according to claim 1, wherein the MSP-1 protein is the MSP-1 protein of the isolate 3D7 or the MSP-1 protein of the FCB 1 strain.

3. The recombinant MVA virus according to claim 1, wherein the fragment is selected from the fragments p83, p30, p38, p33, p 19 and p42 or combinations thereof.

4. The recombinant MVA virus according to claim 1 wherein the mutein is differentiated from the MSP-1 sequence by addition, deletion, insertion, inversion and/or substitution of one or more amino acids.

5. The recombinant MVA virus according to claim 1, wherein the nucleic acid coding for MSP-1 is reduced in its AT content compared to the wild type sequence.

6. The recombinant MVA virus according to claim 1, wherein the nucleic acid coding for MSP-1 is under the control of a promoter.

7. The recombinant MVA virus according to claim 1, wherein the nucleic acid at the 5′ end is fused with a nucleotide sequence coding for a signal peptide sequence.

8. The recombinant MVA virus according to claim 7, wherein the signal peptide sequence controls the secretion of the gene product.

9. The recombinant MVA virus according to claim 7, wherein the signal peptide sequence controls the localisation of the gene product relevant to the membrane.

10. The recombinant MVA virus according to claim 7, wherein the signal sequence controls the GPI anchoring of the gene product.

11. A method of production of a recombinant Modified Vaccinia Vaccine Ankara (MVA) virus wherein the method comprises the steps:
a) transfecting a eukaryotic host cell with a transfer vector, wherein
i) the transfer vector comprises a nucleic acid encoding a Plasmodium falciparum merozoite surface protein-1 (MSP-1) protein, or a fragment or a mutein thereof, wherein the mutein differs by the addition, deletion, insertion, inversion and/or substitution of one or more amino acids from the MSP-1 sequence; and optionally also comprises a selection marker;
ii) the nucleic acid according to i) is flanked by MVA sequences 5′ and/or 3′, wherein the sequences are suitable for the homologous recombination in the host cell;
b) infection with a virus based on MVA, preferably MVA;
c) cultivation of the host cell under conditions suitable for homologous recombination; and
d) isolation of the recombinant virus based on MVA.

12. The method according to claim 11, wherein the virus is isolated from the culture supernatant or from the cultivated host cells.

13. A vaccine comprising:
a) the recombinant virus according to one of the claims 1 to 9; and
b) a pharmacologically compatible carrier.

14. The vaccine according to claim 13, further comprising: c) MSP-1, a fragment or a mutein thereof and/or a nucleic acid coding for MSP-1, or a fragment or mutein thereof.

15. The vaccine according to claim 14, wherein the constituents a) and c) can be administered simultaneously, sequentially or separately.

16. A method for the prophylaxis and/or therapy of malaria, the method comprising administering the recombinant virus of any one of claims 1 to 9.

17. A method for the prophylaxis and/or therapy of malaria, the method comprising administering: i) a recombinant virus according to one of claims 1 to 8; and ii) MSP-1, a fragment or a mutein thereof and/or a nucleic acid coding for MSP-1, or a fragment or mutein thereof.
